(19) United States
(12) Patent Application Publication
(10) Pub. No.: US 2004/0002756 A1
(43) Pub. Date: Jan. 1, 2004

(54) Corrective element for presbyopia

(76) Inventors: Georges Baikoff, Marseille (FR); Scott Hampton, Cumming, GA (US)

Correspondence Address: BAKER BOOTS L.L.P., 44TH FLOOR, 30 ROCKEFELLERAUSTIN PLAZA, NEW YORK, NY, 10112-0228, US

(21) Appl. No.: 10/142,088

(22) Filed: May 9, 2002

(51) Int. Cl. A61F 2/14

(52) U.S. Cl. 623/4.1

(57) ABSTRACT

A corrective element (3) is intended to be implanted in an eye opposite the ciliary body and is shaped to exert on said ciliary body a pressure (F1) directed perpendicular to the optical axis (2) of the eye.

### SPECIFICATION

The present invention concerns, in general, the correction of vision by the insertion of a corrective element in the eye and, more precisely, the correction of presbyopia.

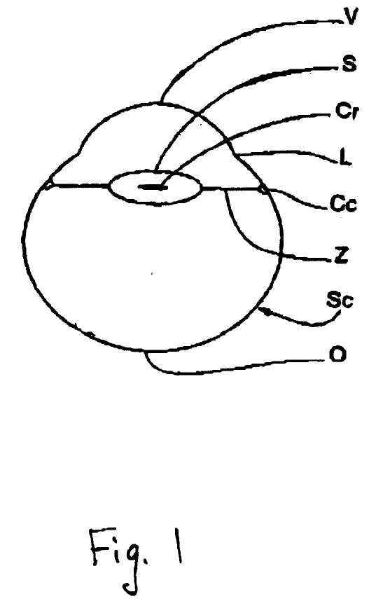

Let us remember that, as represented in FIG. 1, the crystalline lens Cr contained in the crystalline sac S is suspended from the ciliary body Cc by means of the zonular ligament (or zonule). That ciliary body Cc lines the internal face of the sciera along a ring situated in the middle at a latitude 2 to 3 mm away from the limbus, if the optical axis is taken as reference.

Presbyopia is a loss or reduction of the accommodative power of the eye when the subject is aging.

In a theory dating back approximately one hundred years, Von Hehnholtz explained the physiology of accommodation in near vision by a relaxation of zonular tensions exerted on the crystalline lens upon contraction of the ciliary body. That relaxation of tensions results in a resumption by the crystalline lens of a more globular shape presenting lesser and, therefore, more convergent radii of curvature permitting focusing. At the same time, the crystalline lens is displaced forward in an anterior-posterior plane.

For his part, Schachar proposed in 1992, for example, in his U.S. Pat. No. 5,465,737, a theory contrary to that of Von Hehnholtz, according to which accommodation is due to a pull exerted on the crystalline lens when the ciliary body is relaxed, such a pull stretching the zonular ligament, which would create a flattening of the periphery of the crystalline lens and a protrusion of its center.

Furthermore, according to Schachar, the diameter of the crystalline lens increases with aging and the distance separating the periphery of the crystalline lens from the ciliary body gradually diminishes, which leads to a relaxation of the zonule. As a result, the centrifugal force exerted by the ciliary body on the periphery of the crystalline lens is no longer strong enough to ensure accommodation.

Schachar proposed, in the abovementioned patent, different methods making it possible to improve the accommodation power of an eye, consisting, for example, of surgically reducing the length of the zonules or the diameter of the crystalline lens, or preventing enlargement of the crystalline lens.

Another method of treatment proposed by Schachar has been very widely used. It consists of treating presbyopia by positioning a truncated cone-shaped ring around the scleral ring to create a sort of external suspension, in order to enlarge the diameter of the ciliary body and, therefore, restretch the zonule.

Such a procedure being rather cumbersome, it was consequently proposed that arched scleral expansion segments, of radius of curvature less than the radius of curvature of the sclera, be positioned on the sclera. Those segments cross loops incised in the surface of the sclera concentric to the limbus, plumb with the ciliary body, and are supported at their ends on the outer surface of the sclera.

In Applicant's document FR 98 12384, such segments have spatulate ends in order not to risk inducing a perforation of the sclera at the points supporting the segment on the sclera.

It was discovered that the operations carried out since 1992, following Schachar's theory, were sometimes very successful and made it possible to return good vision to the patient, and other times totally failed, accommodation not really be better after the procedure.

On analyzing the results of the procedures practiced by specialists, Applicant came to the conclusion that Schachar's theory would be inaccurate, even though its use makes it possible to obtain the result sought in some cases.

It was therefore concluded that the presbyopia observed in some patients after implantation of a truncated cone-shaped ring or of arched segments on the sclera are not due, as Schachar had thought, to a tensile effect exerted on the zonule and, therefore, on the crystalline lens, but to an induced result obtained on the procedure without the operator seeking it.

Applicant again took into account the fact that the eyeball has a flexible but unstretchable surface and concluded therefrom that it is not possible to increase the circumference of the scleral ring by exerting a centrifugal force at certain points of the sclera.

On examining the procedures performed according to Schachar's method more in detail, Applicant found that on exerting a centrifugal force at certain points, placement of the scleral expansion segments exerted in response a centripetal pressure opposite their points of support on the sciera.

Applicant then explains the effect found with certain rings or scleral expansion segments not by the pull they exert on the zonule at certain points, but by the pressure they exert on the latter at other points. When the pressure is exerted opposite the ciliary body, it artificially compensates for the lack of contraction of said ciliary body and helps the eye in its work of accommodation, as described by Von Helmholtz.

Presbyopia is due to the fact that the crystalline lens grows larger in time, as well as to the fact that the ciliary muscle partially loses its contraction power and to the loss of flexibility of the crystalline lens.

As a result, the method proposed by Schachar becomes effective incidentally, when by chance the ring or the expansion segments exert sufficient pressure on the sclera and when that pressure is exerted precisely on the ciliary body.

Those observations lead Applicant to propose in this invention a corrective element making it possible to correct presbyopia reproducibly and no longer at random, as in the procedures proposed for almost the last ten years.

For that purpose, the invention concerns a corrective element making it possible to correct presbyopia, intended to be implanted in the eye, opposite the ciliary body, characterized in that it is shaped to exert on said ciliary body a pressure directed perpendicular to the optical axis.

That element is remarkable in that it presents in section an outer wall intended to be placed parallel to the surface of the sclera and an inner wall intended to be placed parallel to the optical axis of the eye.

The sclera being unstretchable, such element acts by bearing on the portion of the sclera which covers it in order to push back the ciliary body in the direction of the optical axis of the eye. This results in centripetal forces on said ciliary body artificially compensating for the loss of contraction of the latter and thus restoring its action, necessary for accommodation, that is, a zonular relaxation.

The corrective element according to the invention is further remarkable in that:

it consists of an arched segment whose radius of curvature is such that after its placement in the eye, said segment is centered on the optical axis of said eye,

a back wall connects the separated back ends of said outer and inner walls,

a round connects the concurrent front ends of said outer and inner walls,

the angle between the outer wall and the inner wall is in the order of 45°,

the back wall is rounded,

the back wall is formed by a torus portion.

The invention will be better understood thanks to the specification which follows, given by way of nonlimitative example, with reference to the attached drawings, in which:

FIG. 1 is a schematic cross-section of an eye.

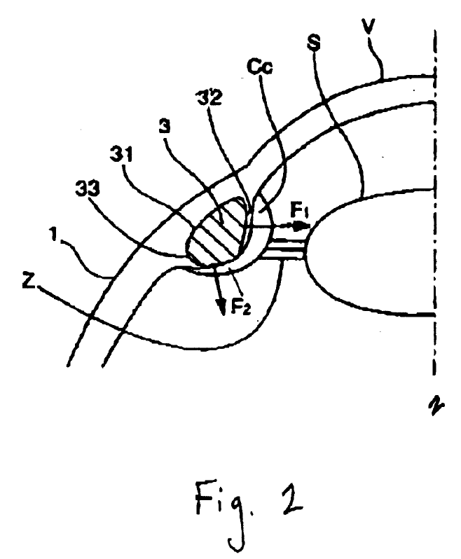

FIG. 2 is a partial cross-section of an eye, in which a corrective element is implanted according to the invention.

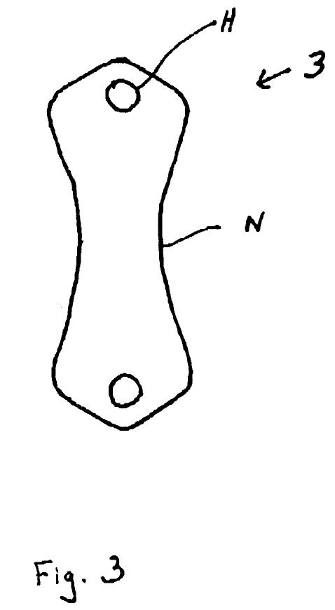

FIG. 3 is a top view of a non-limiting embodiment of the invention having a generally hour glass design.

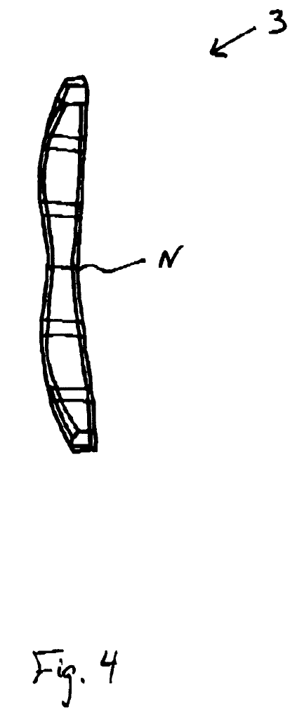

FIG. 4 is a side view of a non-limiting embodiment of the invention having a generally hour glass design.

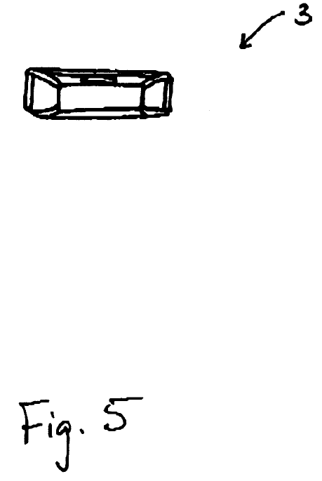

FIG. 5 is a end view of a nonlimiting embodiment of the invention.

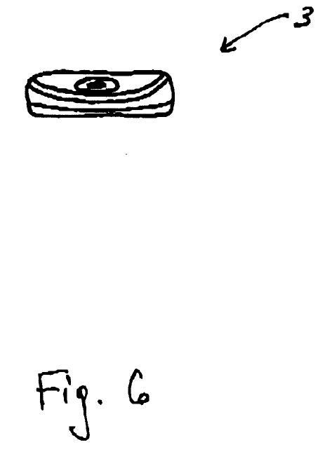

FIG. 6 is an alternative end view of a non-limiting embodiment of the invention.

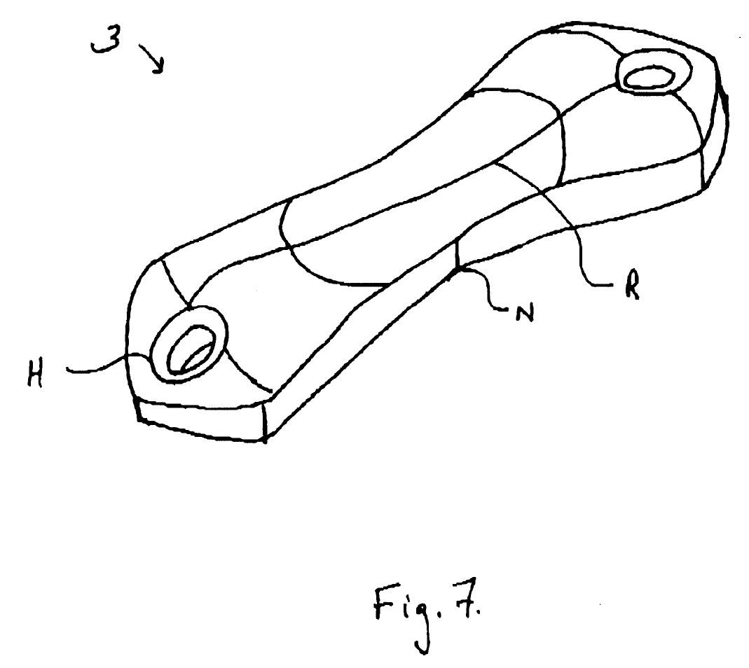

FIG. 7 is a perspective view of a non-limiting embodiment of the invention having an hour glass design.

FIG. 2 the surface of the sclera opposite the ciliary body is represented at 1 and the optical axis of the eye is represented at 2.

The front and back expressions are defined in relation to the eye.

FIG. 2 presents an element 3 according to a preferred embodiment of the invention. That element consists of an arched segment whose radius of curvature is such that it is centered on the optical axis 2 of the eye when it is set in place and, therefore, so that said segment is parallel to the scleral ring at the insertion of the ciliary muscle. Said arched segment has, for example, a length in the order of 4 to 5 millimeters and a radius of curvature in the order of 7 millimeters.

Seen in cross section, the element 3 has generally the shape of a triangle. It contains an outer wall 31 intended to be placed parallel to the surface 1 of the sclera and an inner wall 32 intended to be placed parallel to the optical axis 2 of the eye.

Taking the geometry of the eye into account, that outer wall 31 and inner wall 32 of the element 3 define between them an angle in the order of 45°.

Said outer wall 31 and inner wall 32 have ends concurrent toward the front of the element 3 and separated from each other toward the back.

A back wall 33 connects the separated back ends of the outer wall 31 and inner wall 32 and a round connects the concurrent front ends of the two walls.

When set in place in the eye, opposite the ciliary body, such an element bears at its outer wall 31 on the unstretchable portion of the sclera covering it in order to exert on the ciliary body Cc a force F1 oriented perpendicular to the inner wall 32 and, therefore, perpendicular to the optical axis 2. Such a force acts directly on the ciliary body Cc and pushes it back in the direction of the optical axis of the eye, which artificially creates a displacement of the latter similar to that taking place on a contraction of said ciliary body. That displacement has the effect of relaxing the zonule Z in the axis of the latter. Such effect compensates for the loss of contraction power of the ciliary body and enables the crystalline lens to acquire a more globular share presenting lesser radii of curvature.

The front end of the element 3 is preferably rounded, so as to avoid injury to the tissues of the eye.

The back wall 33 can be arranged to be more or less rounded.

Said back wall 33 is preferably set up to give the section of the corrective element 3 a shape similar to a drop; for that purpose, it can be formed by a torus portion. Such an arrangement makes it possible to further improve the correction generated by the corrective element 3 by supplying a force of pressure F2 exerted on the ciliary body Cc by the back wall 33.

Force F2 is directed toward the center and toward the back of the eye, compressing the vitreous body and inducing a push and, therefore, a displacement of the crystalline lens toward the front, similar to that described by Von Hehnholtz.

FIG. 3 presents an alternative embodiment of the invention wherein the arched segment has an hour glass design. As such, when viewed in a top view the center portion of element 3 has a narrower dimension N than the element at either end. This design assists in maintaining the position of the element after implantation. Optionally, a hole or indentation H at either or both ends of element 3 may be provided to allow for greater control in handling the element upon implantation and may further serve as an egress for suturing the implant in the eye. Illustrated in FIG. 3 is a non-limiting embodiment wherein there is a hole H at both ends of element 3.

FIG. 4 illustrates a side view of the embodiment of FIG. 3 having a further nonlimiting enhancement wherein the center portion of the element has a narrower dimension at the center of the segment in an hour glass design. Even with a tapered center portion, the inner and outer surfaces of the element possess radii of curvature as generally described above.

FIGS. 5 and 6 each illustrate an end view of non-limiting embodiments of the invention, wherein the end has either a squared or rounded design. These non-limiting embodiments are illustrative of other contemplated designs wherein the end of the element is designed in such a way as to avoid injury when inserted into position within the sclera of the eye. In both of these illustrations, an optional hole or indentation is depicted.

FIG. 7 is a non-limiting embodiment of a preferred embodiment of the element having an hour glass shape. As can be seen by the change of curvature, the element is narrower in dimension at the center of the element and greater in dimension at the ends of the element. The element optionally possesses one or more ridges on the surface of the element. As illustrated in FIG. 7, a ridge R runs along the center of the element. Such a ridge also provides for greater ease of handling of the element upon implantation of the device in the sclera of the eye.

The corrective element 3 according to the invention is preferably placed at the deep face of the scleral plane in order to take best advantage of the effects due to the unstretchable nature of the sclera, near the surface of the ciliary body across the small incisions made in the sclera, or between the sclera and the ciliary body, as represented in the drawing.

To treat presbyopia, several segments are distributed in the eye around the ciliary body, in order to constitute support areas distributed around the crystalline lens and induce effects on the latter close to those it bears on contraction of a young ciliary body. For example, from 3 to 8 segments will be positioned according to the invention in the patient's eye and preferably 4 segments placed at 90° from one another.

The corrective element 3 is preferably designed to have in section a length parallel to the optical axis, so that it bears on the ciliary body, even if it is not perfectly well positioned opposite the latter. For that purpose, the length of the inner wall 32 is, for example, in the order of 0.5 to 0.7 millimeter.

The length of the outer wall 31 is, for example, in the order of 3 millimeters and the thickness of segment 3, defined perpendicular to said inner wall 32, is, for example, in the order of 0.5 to 0.7 millimeter.

In an alternative embodiment not represented in the drawing, the corrective element 3 according to the invention consists of a cylindrical ring placed inside the sclera around the ciliary body.

According to still another embodiment not represented in the drawing, the corrective element is not of uniform section over its whole length. It can present triangular section areas acting on the ciliary body and areas of lesser section having only very little or not effect on the ciliary body. Such an embodiment makes it possible to create a large number of support points on the ciliary body in order to better distribute the forces exerted on the latter.

We claim:

1. Presbyopia corrective element intended to be implanted in an eye opposite the ciliary body, characterized in that it is shaped having an hour glass design that exerts on said ciliary body a pressure (F1) directed perpendicular to the optical axis (2) of the eye.

2. Corrective element according to claim 1, characterized in that it presents in section an outer wall (31) intended to be placed parallel to the surface of the sclera (1) and an inner wall (32) intended to be placed parallel to the optical axis (2) of the eye.

3. Corrective element according to claims 1 or 2, characterized in that it consists of an arched segment (3), the radius of curvature of which is such that, after its placement in the eye, said m segment (3) is centered on the optical axis of the eye.

4. Corrective element according to claim 3, characterized in that a back wall (33) connects the separated back ends of said outer wall (31) and inner wall (32).

5. Corrective element according to claim 4, characterized in that a round connects the, concurrent front ends of said outer wall (31) and inner wall (32).

6. Corrective element according to claim 5, characterized in that the angle between the outer wall (31) and the inner wall (32) is in the order of 45°.

7. Corrective element according to claim 4, characterized in that the back wall (33) is rounded.

8. Corrective element according to claim 7, characterized in that the back wall (33) consists of a torus portion.

9. Corrective element according to claim 1, characterized in that the element has an indentation (H) at an end of the element.

10. Corrective element according to claim 1, characterized in that the element has a hole (H) at an end of the element.

11. Corrective element according to claim 1, characterized in that the element has a ridge (R) on the surface of the element.
